(19) United States
(12) Patent Application Publication
(10) Pub. No.: US 2014/0303455 A1
(43) Pub. Date: Oct. 9, 2014

(54) MULTIPURPOSE CEREBROSPINAL FLUID SENSOR ASSEMBLY AND METHOD OF OPERATION OF THE SAME

(71) Applicant: Pharmaco-Kinesis Corporation, Los Angeles, CA (US)

(72) Inventors: Yehoshua Shachar, Santa Monica, CA (US); Thomas Chen, La Canada, CA (US); Dan Murray, El Segundo, CA (US); Robert Purnell, Los Angeles, CA (US)

(73) Assignee: Pharmaco-Kinesis Corporation, Los Angeles, CA (US)

(21) Appl. No.: 14/244,722

(22) Filed: Apr. 3, 2014

Related U.S. Application Data
(60) Provisional application No. 61/808,103, filed on Apr. 3, 2013.

(51) Int. Cl. A61B 5/00; A61B 5/145; A61B 5/103; A61B 5/01; A61B 5/03

(52) U.S. Cl. CPC A61B 5/4851; A61B 5/686; A61B 5/01; A61B 5/031; A61B 5/1032; A61B 5/0022; A61B 5/14532; A61B 5/746
USPC 600/301

(57) ABSTRACT

A cerebrospinal fluid (CSF) monitoring device for the detection of obstructions in real time through the collection of pressure data from an on-board pressure sensor. This system is designed to integrate into medical devices equipped with a CSF drainage system, such as implantable shunts. CSF analysis data can be transmitted in real-time wirelessly to a physician or can be stored on-board in a memory card and retrieved wirelessly at a later time. Information from the CSF sensors can be used by physicians to monitor CSF conditions known to cause shunt malfunction/obstruction such as elevated protein concentrations, evidence of an on-going or recent hemorrhage such as presence of heme proteins, and concentrations of administered medications/drugs over time. It can be used to monitor for CSF infections, in which increased pressure, increased temperature, CSF high protein and low glucose, would be suggestive of CSF infections.

### RELATED APPLICATIONS

The present application is related to U.S. Provisional Patent Application Ser. No. 61/808,103, filed on Apr. 3, 2013, which is incorporated herein by reference and to which priority is claimed pursuant to 35 USC 120.

### BACKGROUND

1. Field of the Technology

This invention generally relates to implantable and external monitoring devices and related methods. More specifically, this invention relates to such devices and methods useful in monitoring cerebrospinal fluid (CSF) physical parameters and constituent concentrations in patients with implanted CSF drainage systems and/or infusion pumps (implanted or external), and methods in detecting obstructions in drainage systems and/or infusion pumps, and identifying abnormalities in CSF physical parameters related to particular health conditions.

2. Description of the Prior Art

Cerebrospinal Fluid (CSF)

Cerebrospinal fluid (CSF) is a physiological fluid contained in the brain ventricles and subarachnoid space to protect and mitigate the effect of shocks on the brain. CSF is continuously produced in the ventricles at a rate of approximately 500 mL per day, and the entire volume (approximately 135 mL in adults) is replaced every six hours, as it gets reabsorbed by the body. In normal, healthy individuals, the rate of CSF production (0.35 mL/min.) is comparable to the rate of re-absorption. At equilibrium, the CSF exerts a positive intracranial pressure (ICP) in the 0-15 mmHg range, depending on many factors, such as the body position (standing or lying down) and recent physical activity. If CSF is not properly reabsorbed, often due to blockages in the ventricles, then ICP can build up and even reach life threatening levels. Generally, when ICP is over 15-20 mmHg, it needs to be reduced by intervention.

Hydrocephalous

Hydrocephalous is a medical condition in which there is an abnormal accumulation of cerebrospinal fluid (CSF) in the brain ventricles and/or cavities. Excess CSF causes elevated ICP, which may cause enlargement of the skull, neurological damage, and even death. Hydrocephalus is often treated by implanting a CSF shunt.

Shunts

Cerebrospinal fluid shunts are drainage systems commonly used to treat individuals affected by hydrocephalus. Shunt systems are typically comprised of a ventricular catheter, a shunt valve, and a drainage catheter (also called a distal catheter). The ventricular catheter is placed into the brain through a hole in the skull and positioned into the brain ventricles. The catheter provides a means for excess CSF to be relieved, thus maintaining ICP within normal ranges. The upper level of allowed ICP is regulated by a mechanical valve. Excess CSF drained through the catheter is released in the body, typically in the peritoneal cavity or in the atrium, where it can be absorbed.

Complications with Shunts

There are two main complications that can occur with shunts: obstruction and infections. These potential issues need to be considered every time a shunt has to be implanted to relieve a patient from high ICP. These issues continue to affect a significant portion of the treated population. For instance, shunt obstruction and malfunction are the most common causes of shunt failure, and occur in approximately one third of pediatric patients within the first year. Shunt failures or suboptimal performance are difficult to diagnose, as they are caused by a number of factors. The next section discusses some of these factors, including high levels of CSF proteins and bacterial infections.

Problems with High Levels of CSF Proteins

Higher than normal CSF protein levels can cause shunt malfunction and obstruction as these proteins coagulate around the ventricular catheter tip or at the shunt valve. A number of brain conditions can cause elevated proteins, such as hemorrhage, infections, blood brain barrier (BBB) dysfunction, or simply high protein content in the blood/serum. Normal levels of total protein are in the 0.15-0.45 mg/mL range. Because the concentration of proteins in serum is about one hundred times higher than in CSF in healthy individuals, any hemorrhage or BBB dysfunction can cause elevated CSF proteins, and thus increase the risk of a shunt obstruction. Patients with CSF infections (i.e. bacterial meningitis) have on average double or triple the proteins levels. Currently, accessing the CSF to determine the concentration of total proteins requires at least a lumbar puncture or shunt tap and a sample sent off to a laboratory. A device and a method to monitor, in-vivo and in real-time, increasing trends of total proteins in CSF would be very valuable.

Problems with Blood Brain Barrier (BBB) Dysfunctions

Dysfunction of the BBB can result in higher CSF proteins (especially albumin), as blood proteins can more easily cross the BBB. A BBB dysfunction might be temporary, for instance due to the assumption of certain medications/drugs, or might be prolonged. When the BBB dysfunction is prolonged, there is a higher chance that high CSF protein levels are persistent, resulting in a higher probability of obstruction. It would be very valuable for a treating physician to determine if a change in the total protein level is merely a temporary fluctuation or if it is a prolonged/semi-permanent condition.

Problems with Hemorrhage

Hemorrhages and hematomas are also important conditions that can potentially cause shunt malfunctions. If whole blood drips into CSF, it will carry fibrogen, the clotting protein, which can facilitate the clogging of the catheter tip or shunt valve and cause a malfunction. Beside the dangers associated with causing obstruction, there should never be any blood in CSF. Thus, it would be very valuable for a treating physician to have a tool and a method for identifying an on-going hemorrhage in-vivo and in real-time.

Problems with Infections

A major problem associated with shunts for hydrocephalous patients, besides obstruction, is the involuntary inoculation of cerebrospinal fluid (CSF) with viable bacterial cells. Post-operative CSF infections occur in 5-8% of the approximately 70,000 shunt-related procedures performed each year in the U.S. When a shunt infection occurs, the shunt has to be removed and antibiotics given until the CSF clears up. Therefore, it is crucial that a timely and accurate diagnosis be made. Currently, in order to diagnose a shunt infection, CSF needs to be accessed from the shunt and sent off to culture. Other useful biological/physical parameters aiding the diagnosis of shunt contaminations include an elevation in systemic white blood cell count, leukocyte count of over 250 cells per μL of CSF (healthy control count is less than 5 cells/μL), increased intracranial pressure (ICP), low levels of glucose due to high cellular metabolism, and possibly change in CSF color from clear to yellowish. The ability to monitor, in-vivo, these biological/physical signs of shunt infections via an implanted sensor, without having to repeatedly tap a shunt, can provide valuable information on whether there is an actual shunt infection and/or on the stage of the infection (early stage or advanced), thus reducing the time, cost, and morbidity of repeat shunt taps.

What is Lacking in Current Shunt Systems

While shunts are a clinically proven technology that improved the quality of life of millions of hydrocephalus patients, they remain a “primitive” technology in the sense that they lack any of the integrated tools and methods used to predict and then diagnose malfunctions and infections.

Continuousness of Monitoring

Accurate prediction and diagnosis would require monitoring CSF in-vivo and in real-time for signs of possible causes of obstruction, malfunction, and infection. The ability to perform these functions at home and in the clinic is critical to identify issues before significant side-effects occur.

Comparing to Baseline Signals

To determine if the properties of CSF are normal or abnormal, measurements have to be compared to baseline values for the same patient, because constituents can vary significantly with race, diet, age, gender, etc. However, these baseline values are difficult to establish because CSF measurements, while simple to obtain from an analytical point of view, are not practical or cost effective to get because of the low-accessibility of CSF.

What is Specifically Needed to Make Shunts Safer

An integrated tool and a method to detect obstructions that lead to failure would lead to an improvement in the quality of life for the thousands of hydrocephalus patients with implanted CSF shunts. The same device that can monitor for infection in the CSF will prevent unnecessary shunt taps.

Monitoring of CSF In-Vivo and Real-Time

The shunt monitoring system should: be able to record a history of baseline signals that correlate with normal levels of protein concentration and internal pressure in the CSF; recognize changes in these baseline signals indicative of serious complications in the shunt (e.g., malfunction due to infection or obstruction); operate remotely, only when fresh sample of CSF have been drained (<1 hour/day) to save power; discriminate between fresh and abnormal CSF protein concentration; and monitor ICP and indicate when pressure reaches the safety threshold of 18 mmHg. The implantable system will also be able to measure CSF glucose levels (which goes down in infections) and temperature (which increases when infections are present).

Implantable and External Infusion Pumps

Infusion pumps are medical devices used to infuse fluids, medication, or nutrients into a patient's circulatory system. Certain types of infusion pumps are used to deliver fluids into CSF. Other types of pumps are used to regulate the lumbar subarachnoid drainage of CSF, often in conjunction with the treatment of CSF fistula and control of intracranial pressure in children and adults. Whether implanted or external, infusion pumps can deliver precise amounts of fluids over a long period of time. Some models of infusion pumps are equipped with a drainage or a sampling lumen/system, where fresh CSF flows through directly from the central nervous system. The functionality of any pump can be monitored by connecting the shunt monitoring system at any collection or infusion point in direct contact with the CSF.

Problems with Infusion Pumps

Infusion pumps are also a potential source of problems. Especially in implantable pumps, if the CSF is accessed using a ventricular catheter, then there is always a chance of obstruction, malfunction, and infection similar to shunt systems. A shunt monitoring system could also be used in conjunction with infusion pumps (implanted and external) to detect an increase in protein concentration and ICP associated with infection and obstruction of fluid lines in the pump.

What is Lacking in Implanted Infusion Pumps

Current infusion pumps, similarly to shunts, lack integrated analytical systems to 1) detect and eliminate obstructions that form in fluid lines and 2) provide feedback to the treating physician.

The illustrated embodiments of the shunt monitoring system address all the above mentioned issues with shunts and infusion pumps.

### BRIEF SUMMARY

The illustrated embodiments of the invention provide an implantable shunt monitoring device that provides real-time measurements of the ICP and protein concentration of cerebrospinal fluid (CSF) and detects any obstructions through application of an internal pressure sensor. CSF is a physiological fluid contained in the brain ventricles and subarachnoid space, and is produced continuously in the ventricles at a rate of approximately 0.35 mL/min. Normally, the rate of CSF production is comparable to the rate of re-absorption. When not properly reabsorbed, the “trapped” CSF significantly increases the ICP. Hydrocephalous, a medical condition characterized by a high ICP due to abnormal CSF accumulation in the brain ventricles and/or cavities, is often treated by implanting a CSF shunt. The purpose of the shunt is to discharge excess CSF and reduce the ICP to normal levels. While this is an effective countermeasure for hydrocephalus, the obstruction and malfunction of shunts leads to an increase in elevated ICP with additional risks of bacterial infection. Shunt failure is predominantly associated with high levels of CSF proteins, which can be attributed to a number of conditions, including blood brain barrier dysfunctions, hemorrhages, high serum proteins, and infections (e.g., bacterial or fungal). Current shunts lack any integrated tools and methods to detect any malfunctions due to formation of an obstruction that occurs with infection. Accurate detection of a clog would require: (1) continuously monitoring the CSF for signs of conditions known to leading to malfunctions and infections; (2) determining if the concentrations of certain CSF constituents is normal or abnormal when compared to the patient's personal baseline values.

The components of the shunt monitoring system address all the above mentioned issues related to shunts and infusion pumps. The CSF monitoring shunt system is a stand-alone implantable device that integrates into implantable shunts or infusion pumps (implanted or external) and utilizes their drainage systems to directly monitor ICP. The existing shunt system is left entirely intact. The only difference is that the CSF monitoring system is spliced into the peritoneal tubing. The fluid line includes an inline pressure sensor, a spectrophotometer, a glucose sensor, and a temperature monitor. If the shunt is working properly, the valve will regulate the flow of fluids past a certain intracranial pressure. If the shunt is blocked proximally (i.e. at the level of the ventricular catheter), the pressure sensor will read a low pressure. The spectrophotometer will help determine if infections are present by collecting absorption spectra in the line. The resulting data set from each measurement can be transmitted in real-time wirelessly to a physician or can be stored on-board in a memory card and retrieved wirelessly at a later time.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is an illustration depicting how the current device is be placed into a patient and incorporated into an existing cerebrospinal fluid drainage system.

FIG. 2 is a schematic diagram of the internal components of the shunt monitoring system of the current invention.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

The illustrated embodiments of the invention provide the shunt monitoring system 1 to be utilized in conjunction with other medical devices and systems accessing patients' cerebrospinal fluid, such as: CSF drainage systems (“shunts”), and any other medical devices that has direct access to the patient's CSF. The shunt monitoring system 1 monitors the drained CSF in-vivo (in implanted devices) and in real-time, and creates a profile specific to each patient. The shunt monitoring system 1 aids in the identification of abnormalities for a given CSF scan or a sequence of CSF scans when compared to the patient CSF baseline profile. In one embodiment, the shunt monitoring system 1 is comprised of materials that are fully compatible with standard sterilization procedures.

Any patient treated with a medical device equipped with any type of CSF drainage system, which can access and sample his/her CSF, can benefit from the shunt monitoring system 1. In one example, the population benefitting from this invention includes hydrocephalous. In another example, the population benefitting from this invention includes patients receiving treatment via infusion pumps.

For the purposes of ease of description, “proximal” refers to the end or portion of the catheter which located near the brain of the patient, while “distal” refers to the end or portion of the catheter that is located near the peritoneal cavity or atrium of the patient.

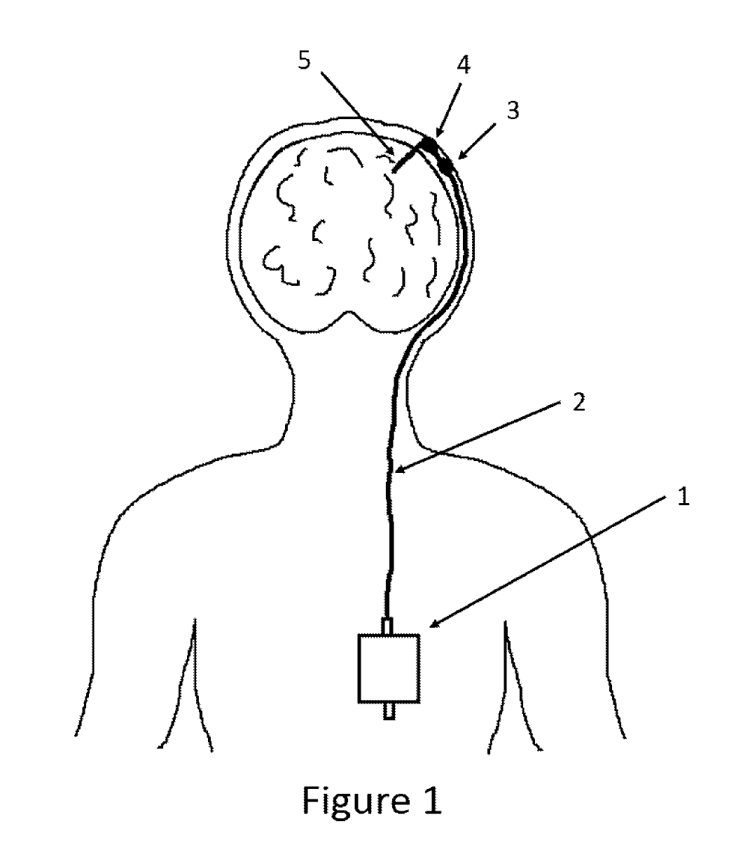

In one embodiment seen in FIG. 1, the shunt monitoring system 1 is incorporated into a pre-existing shunt drainage configuration. A proximal or ventricular catheter 5 is inserted into the brain ventricle of a patient as is known in the art. A valve 3 and a septum access port 4 are coupled to the ventricular catheter 5 which are turn coupled to a distal catheter 2. The distal end of the distal catheter 2 terminates in the shunt monitoring system 1 which is implanted within the patient. An outlet is provided on the shunt monitoring system 1 which allows access to the peritoneal cavity or the atrium of the patient, where excess CSF can be released and reabsorbed by the body.

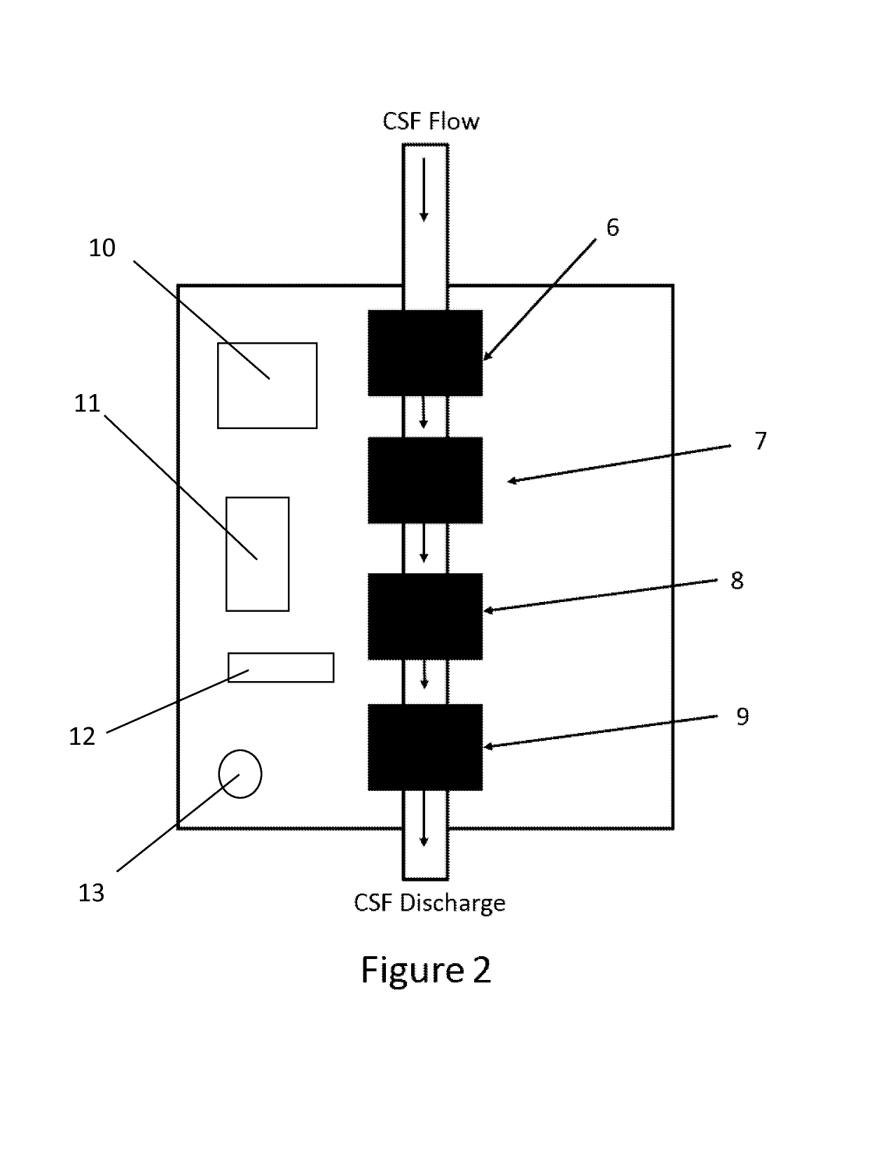

FIG. 2 is a magnified schematic view of the internal components of the shunt monitoring system 1. The shunt monitoring system 1 comprises a main flow channel in which the CSF flows through. The CSF flows through a pressure sensor 6, a spectrophotometer 7, a glucose monitor 8, and finally a temperature sensor 9 in sequential order. It is to be expressly understood however that different or additional sensors not shown or described in FIG. 2 may also be incorporated within the shunt monitoring system 1 without departing from the original spirit and scope of the invention. Additionally, the specific placement of the plurality of sensors may different from what is actually shown in FIG. 2 without departing from the scope of the invention. For example, the sequential order of the placement of the sensors may be rearranged or reconfigured to fit the specific needs of a patient or to the specific treatment regimen implemented by a physician. After passing through the plurality of sensors 6-9, the CSF then exits the shunt monitoring system 1 and continues into the peritoneum.

The shunt monitoring system 1 is equipped with a power source, such as a rechargeable battery 11. In one embodiment, the size of the battery 11 is specifically chosen to fit into a small space. In another embodiment, in the case of implanted devices (shunts and certain infusion pumps), the battery 11 can be recharged by inductive coupling as in known in the art.

The shunt monitoring system 1 can communicate data wirelessly with an external processing unit. In one embodiment, a wireless communication system or wireless radio transmitter 13 can transmit collected data from the plurality of sensors, information regarding the lifetime of the battery, and alarm signals if something abnormal is detected. In another example, this wireless communication system 13 can receive inputs from the treating physician or programmer, for instance to change the schedule of a measurement and/or to retake a measurement.

The shunt monitoring system 1 comprises at least one integrated memory chip 10 to store information regarding analytical modes, the date, programs, software, instructions, etc. which may be wirelessly transmitted to the shunt monitoring system 1 by the physician or programmer.

In another embodiment, the CSF shunt monitoring system 1 is equipped with an internal clock 12, set remotely and synchronized with computers external to the shunt monitoring system 1. This clock 12 marks the data collected with a day/time stamp. This clock 12 is essential to build a baseline of CSF profiles.

The enclosure of the CSF shunt monitoring system 1 is made of a thermally insulating material to avoid localized overheating in the patient's body, whenever the shunt monitoring system 1 is implanted. The shunt monitoring system 1 can be controlled remotely by a qualified physician or programmer.

In another embodiment, the CSF shunt monitoring system 1 can monitor the ICP and protein concentration on a regular schedule set by the treating physician and communicated wirelessly to the shunt monitoring system 1. For example, the CSF shunt monitoring system 1 can monitor the CSF profile whenever a fresh volume of CSF passes through the device.

The shunt monitoring system 1 can be used to record a patient's CSF profiles (i.e. glucose or protein) as a function of time, so an accurate history or baseline can be determined. This baseline includes all the parameters measurable with the sensor. This CSF baseline profile is critical to identify abnormalities in future CSF scans or sequences of scans, and permanent trends can be identified and distinguished from temporary fluctuations. The profile includes all the parameters that can be monitored on a regular schedule in order to establish a history of baseline levels. Such parameters include the ICP and the total CSF protein concentration.

The shunt monitoring system 1 records changes in the ICP and the concentration of protein and glucose simultaneously. These data points are analyzed by software and reported in a CSF profile. Moreover, data are immediately compared to baseline values previously recorded. This data presentation in a single profile present a clear advantage over previous lab analysis, where different CSF samples are analyzed at different times and CSF parameters are reported separately, making the entire CSF profile more difficult to visualize and interpret.

The shunt monitoring system 1 can identify and distinguish unsafe trends from temporary innocuous fluctuations. For example, the shunt monitoring system 1 can identify trends in the CSF profile that can lead to unsafe conditions, such as trends of increasing proteins, and increasing frequency of development of obstructions.

The shunt monitoring system 1 can send out warning or alarm signals, throughout a wireless communication network, to the treating physician when processed data and simulations indicate a suspicious trend in the most recent CSF scans. The treating physician can then closely monitor the patient, request further labs, or even intervene immediately.

The CSF shunt monitoring system 1 can aid a treating physician in the identification of abnormalities in the CSF profile that relate to suspicious or unsafe health conditions, monitor and understand the pharmacokinetics and pharmacodynamics of infused medications, and/or monitor the efficacy of a therapy. For example, the shunt monitoring system 1 can alert the physician if an obstruction is occurring repeatedly or cannot be removed through hydrostatic pressure. Additionally, the UV-Vis spectrophotometer 7 can be used to measure the CSF optical density, estimate the amount of total proteins in CSF, spot traces of heme proteins or decomposing heme proteins, and determine if light scattering “objects” such as human cells, bacterium cells, or debris are present in amounts higher than what is present in a normal or baseline sample of CSF.

In another embodiment, the UV-Vis spectrophotometer 7 alerts the physician if trends of increasing total proteins are observed in a sequence of CSF scans. Positive trends of total protein are a sign of suspicious or dangerous health conditions. The UV-Vis spectrophotometer 7 can also alert the physician if persistent levels of total proteins are observed, as these high levels are often correlated to subsequent shunt obstructions and malfunctions.

In another embodiment, the UV-Vis spectrophotometer 7 alerts the physician through an alarm circuit if signs of an ongoing hemorrhage are observed in a CSF scan. Such signs would include high proteins, high cells (including red blood cells), and the characteristic absorption signature of hemoglobin and heme proteins. The UV-Vis spectrophotometer 7 further alerts the physician through an alarm circuit if signs of a recent hemorrhage are observed in a CSF scan. Such signs would include the specific absorption signature of oxidized or decomposing heme proteins, a condition known as bilirubin.

Additionally, the pressure sensor 6 can be used to monitor the pressure inside the flow channel and monitor for signs of obstructions. The glucose monitor 8 is used to monitor the glucose concentration in the CSF. Low glucose suggests an infectious process. Finally, the temperature sensor 9 is used to monitor the temperature of the CSF. Increased temperature is a suggestive indication of an infectious process.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A shunt monitoring system for use with cerebrospinal fluid drainage systems in a patient, the shunt monitoring system comprising:
a flow channel disposed through the system in which cerebrospinal fluid (CSF) is optically analyzed by a plurality of sensors, wherein the plurality of sensors comprise;
a pressure sensor for measuring the intracranial pressure (ICP) of the patient;
a glucose sensor; and
a temperature sensor;
an integrated memory chip for archiving recorded sensor data disposed in the system and coupled to the suite of sensors;
an integrated wireless radio transmitter coupled to the integrated memory chip; and
a battery coupled to the suite of sensors, the wireless radio transmitter, and the integrated memory chip.

2. The monitoring system of claim 0 where the monitoring system comprises means for being used in a conventional shunt system and is coupled to a distal catheter of the shunt system and is implanted into the peritoneum of the patient.

3. The monitoring system of claim 0 where the plurality of sensors monitor the CSF of the patient in real-time and in-vivo.

4. The monitoring system of claim 1 where the plurality of sensors further comprise a spectrophotometer for measuring the optical density and color/pigmentation of the CSF.

5. The monitoring system of claim 1 where the flow channel is comprised of a low flow-resistance, biocompatible material.

6. The monitoring system of claim 1 where the flow channel has the same inner diameter as the catheter, shunt valve opening, or drainage shunt terminal tip used in the cerebrospinal fluid drainage system in the patient in order to avoid CSF flow changes.

7. The monitoring system of claim 1 where the pressure sensor measures pressures in range of 3-20 mm Hg of positive pressure within an accuracy of 0.1 mm Hg.

8. The monitoring system of claim 1 where the pressure sensor is disposed inside the flow channel where the pressure sensor monitors for signs of obstructions inside the monitoring system.

9. The monitoring system of claim 1 where the pressure sensor is disposed in an upstream portion of the flow channel to aid in the identification of signs of obstructions inside the flow channel.

10. The monitoring system of claim 0 where the plurality of sensors, the flow channel, the integrated memory chip, and the battery are comprised of sterilized and biocompatible materials.

11. The monitoring system of claim 0 where the integrated wireless radio transmitter comprises means for communicating data wirelessly with an external processing unit.

12. The monitoring system of claim 1 further comprising a check valve to prevent the back flow of other body fluids into the monitoring system.

13. The monitoring system of claim 0 further comprising an alarm circuit coupled to the suite of sensors for generating warning or alarm signals.

14. A method for monitoring a cerebrospinal fluid (CSF) drainage system in a patient comprising:
directing a flow of CSF through a flow channel;
measuring a plurality of physical parameters of the CSF using a plurality of sensors disposed within the flow channel;
recording the measured parameters in an integrated memory chip coupled to the plurality of sensors; and
transmitting the recorded parameters wirelessly to an external processing unit.

15. The method of claim 14 where measuring the plurality of parameters of the CSF comprises:
identifying abnormalities in the physical characteristics of the CSF; and
detecting any obstructions in the shunt.

16. The method of claim 14 where measuring the plurality of parameters of the CSF measuring the protein concentration within the CSF of the patient.

17. The method of claim 14 where recording the measured parameters comprises recording a measured intracranial pressure and a protein concentration value obtained by the plurality of sensors and forming a baseline profile for each measured parameter.

18. The method of claim 14 where measuring the plurality of parameters of the CSF comprises:
measuring parameter conditions associated with CSF infections;
measuring parameter conditions associated with on-going hemorrhaging or recent hemorrhaging;
measuring parameter conditions associated with blood brain barrier (BBB) dysfunction; and
measuring parameter conditions associated with a response to treatment.

19. The method of claim 15 where identifying abnormalities in the physical characteristics of the CSF comprises measuring the intracranial pressure (ICP) of the patient and non-invasively detecting a potential shunt infection while minimizing the need for tapping a shunt.

20. The method of claim 14 where measuring a plurality of physical parameters of the CSF using a plurality of sensors disposed within the flow channel comprises detecting the presence of cells alien to normal and healthy sample of the patient's CSF, including red blood cells, bacterial cells, and/or cancer cells.

21. The method of claim 14 where measuring a plurality of physical parameters of the CSF using a plurality of sensors disposed within the flow channel comprises measuring the absorption or transmission of light through the CSF at one or more wavelengths using a UV-Vis spectrophotometer.

22. The method of claim 21 where the measured data obtained by the UV-Vis spectrophotometer is used for:
estimating the amount of total proteins in CSF;
detecting heme proteins or decomposing heme proteins; or
determining if light scattering objects such as human cells, bacterium cells, or debris are present in amounts higher than a predetermined baseline amount.

23. The method of claim 21 where the measured data obtained by the UV-Vis spectrophotometer is used for deriving the concentration of a drug molecule in the CSF.

24. The method of claim 14 where measuring a plurality of physical parameters of the CSF using a plurality of sensors disposed within the flow channel comprises measuring a CSF parameter during hypothermia treatment, head trauma treatment, or any procedure requiring monitoring of the intracranial pressure (ICP).

25. A method for monitoring the response to a pharmacological therapy performed on a patient comprising:
performing the pharmacological therapy to the patient;
measuring a plurality of physical parameters of the cerebrospinal fluid (CSF) of the patient; and
analyzing the measured parameters of the CSF and its constituent concentrations.

26. The method of claim 25 where analyzing the measured parameters of the CSF comprise analyzing and fitting the measured parameters related to CSF to evaluate the response to the therapy using modeling software.
